(19) United States
(12) Patent Application Publication
(10) Pub. No.: US 2012/0141584 A1
(43) Pub. Date: Jun. 7, 2012

(54) Multilayer Minitablets

(75) Inventors: Ishwar Chauhan, Voorhees, NJ (US); Siva Rama Krishna Nutalapati, Princeton, NJ (US)

(73) Assignee: Aptapharma, Inc., Pennsauken, NJ (US)

(21) Appl. No.: 13/389,641

(22) PCT Filed: Aug. 13, 2010
(86) PCT No.: PCT/US10/45414

§ 371 Date: Feb. 9, 2012

Related U.S. Application Data
(60) Provisional application No. 61/237,018, filed on Aug. 26, 2009.
(60) Provisional application No. 61/251,075, filed on Oct. 13, 2009.

(51) Int. Cl. A61K 9/52; A61K 9/48; A61P 29/00; A61K 9/28

(52) U.S. Cl. 424/457; 424/472; 424/474; 424/451

(57) ABSTRACT

Multilayer minitablets for oral administration of an active pharmaceutical ingredient which release the active pharmaceutical ingredient at different pH ranges and/or in different release profiles are described.

This patent application claims the benefit of priority from U.S. Provisional Application Ser. No. 61/251,075, filed Oct. 13, 2009 and U.S. Provisional Application Ser. No. 61/237,018, filed Aug. 26, 2009, teachings of each of which are herein incorporated by reference in their entirety.

### FIELD OF THE INVENTION

The present invention relates to multilayer minitablets 5 mm or less in diameter for oral administration of an active pharmaceutical ingredient. The minitablets are formulated so that the layers release the active pharmaceutical ingredient at different pH ranges and/or at different release rates. These minitablets are formulated into capsules for oral administration of one or more active pharmaceutical ingredients.

### BACKGROUND OF THE INVENTION

Controlled release formulations for active pharmaceutical ingredients such as proton pump inhibitors are described in, for example, published U.S. Applications 2006/0177509, 2009/0104264 and 2009/0175959.

### SUMMARY OF THE INVENTION

An aspect of the present invention relates to a multilayer minitablet 5 mm or less in diameter formulated so that the different layers release an active pharmaceutical ingredient at different pH ranges and/or at different release rates.

In one embodiment, the multilayer minitablet of the present invention comprises one or more immediate release layer(s) containing an active pharmaceutical ingredient, one or more modified release layer(s) containing an active pharmaceutical ingredient compressed with or to the immediate release layer(s) to form a core minitablet, and a pH dependent enteric coating around the core minitablet. The minitablet may further comprise optional inert layers to further control release of the active pharmaceutical ingredient from the modified release layer(s).

In this embodiment, the minitablet may further comprise an optional subcoating and/or functional coating between the core minitablet and the pH dependent enteric coating.

In another embodiment, the multilayer minitablet of the present invention comprises one or more immediate release layers or one or more modified release layers compressed to form a core minitablet. Each immediate release layer or each modified release layer contains an active pharmaceutical ingredient. The compressed core minitablet is then coated with a pH dependent enteric coating.

In this embodiment, the minitablet may further comprise an optional subcoating and/or functional coating between the core minitablet and the pH dependent enteric coating.

In another embodiment, the multilayer minitablet of the present invention comprises a single or multilayer core minitablet containing an active pharmaceutical ingredient, a first enteric coating which dissolves at a pH of 6.8 or higher on the core minitablet, a second active pharmaceutical ingredient containing layer coated on the first enteric coating and a second enteric coating which dissolves at a pH of 3.5 or higher on the second active pharmaceutical ingredient containing layer.

In this embodiment, the minitablet may further comprise an optional subcoating and/or functional coating between the active pharmaceutical ingredient containing layer and the pH dependent enteric coating.

In yet another embodiment of the present invention, the multilayer minitablet comprises a first active pharmaceutical ingredient compressed into a single or multilayer core minitablet. A second active pharmaceutical ingredient containing layer is then coated on the single or multilayer core minitablet. An enteric coating which dissolves at a pH of 3.5 or higher is coated on the second active pharmaceutical ingredient containing layer.

In this embodiment, the minitablet may further comprise an optional subcoating and/or functional coating between the first and second active pharmaceutical ingredient containing layers and/or the pH dependent enteric coating.

### DESCRIPTION OF THE FIGURES

FIG. 1 is a diagram of one embodiment of a multilayer minitablet of the present invention. In this embodiment, the minitablet comprises an immediate release layer containing an active pharmaceutical ingredient and a modified release layer containing an active pharmaceutical ingredient compressed thereto to form a multilayer core minitablet. The core minitablet is then coated with a pH dependent enteric coating. As shown in this embodiment, a subcoating (or functional coating) can be applied to the compressed layers prior to application of the pH dependent enteric coating.

FIG. 2 is a diagram showing another embodiment of a multilayer minitablet of the present invention. In this embodiment, the core minitablet comprises a single or multilayer minitablet with a first active pharmaceutical ingredient, a first enteric coating which dissolves at a pH of 6.8 or higher applied to the core minitablet, a second active pharmaceutical ingredient containing layer applied to the first enteric coating, and a second enteric coating which dissolves at a pH of 3.5 or higher applied to the second active pharmaceutical ingredient containing layer. In this depicted embodiment, an optional subcoating (or functional coating) is applied between each active pharmaceutical ingredient containing layer and the pH dependent enteric coating.

FIG. 3 is a diagram showing an embodiment of a core minitablet of a multilayer minitablet of the present invention. In this embodiment, the core minitablet comprises one or more immediate release layer(s) compressed with one or more modified release layer(s). The minitablet further comprises optional inert layers to further control release of the active pharmaceutical ingredient from the modified release layer(s).

As will be understood by the skilled artisan upon reading this disclosure, multilayer minitablets can comprise only immediate release layer(s) or only modified release layer(s) containing an active pharmaceutical ingredient. Additional inert layers can be added to the core tablet and minitablets comprising these additional layers are encompassed by the present invention.

### DETAILED DESCRIPTION OF THE INVENTION

The present invention relates to multilayer minitablets 5 mm or less in diameter for oral administration of an active pharmaceutical ingredient. The minitablets are formulated so that the different layers release an active pharmaceutical ingredient at different pH ranges and/or at different release rates. The core minitablet may comprise a single orally administered drug or multiple orally administered drugs.

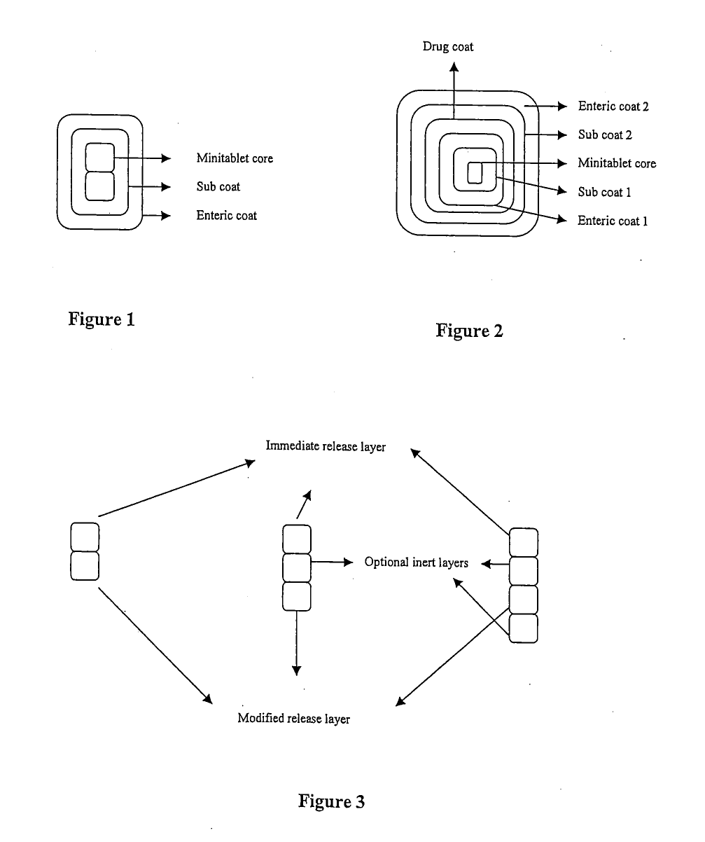

In one embodiment of the present invention, as depicted in FIG. 1, the multilayer minitablet comprises a bilayer core tablet with an immediate release layer containing an active pharmaceutical ingredient and a modified release layer containing an active pharmaceutical ingredient compressed with or to said first immediate release layer. This bilayer core tablet is then coated with a pH dependent enteric coating around the bilayer core tablet. In this embodiment, the minitablet may further comprise an optional subcoating and/or functional coating between the core tablet and the pH dependent enteric coating.

In this embodiment, the minitablet may further comprise optional inert layers (as depicted in FIG. 3) to further control release of the active pharmaceutical ingredient from the modified release layer(s). By “inert layer” it is meant a layer containing no drug or drug in an amount which is insignificant to therapeutic activity of the minitablet or formulations thereof.

In another embodiment, the multilayer minitablet of the present invention comprises one or more immediate release layers or one or more modified release layers compressed to form a core minitablet. Each immediate release layer or modified release layer contains an active pharmaceutical ingredient. The compressed core minitablet is then coated with a pH dependent enteric coating. In this embodiment, the minitablet may further comprise an optional subcoating and/or functional coating between the core minitablet and the pH dependent enteric coating.

As will be understood by the skilled artisan upon reading this disclosure, additional immediate release layers and/or modified release layers containing an active pharmaceutical ingredient and/or additional inert layers can be added to the core tablet and minitablets comprising these additional layers are encompassed by the present invention. Addition of such layers in no way circumvents the present invention.

In another embodiment of the present invention, a single or multiple layer core minitablet is compressed with first active pharmaceutical ingredient. In case of a single layer minitablet, it can be formulated either as an immediate release minitablet or as a modified release minitablet. This core minitablet is then coated with a first enteric coating which dissolves at a pH of 6.8 or higher. A second active pharmaceutical ingredient containing layer is then applied over top of the first enteric coating followed by a second enteric coating which dissolves at a pH of 3.5 or higher over top of the second active pharmaceutical ingredient containing layer.

In yet another embodiment of the present invention, the multilayer minitablet comprises a first active pharmaceutical ingredient compressed into a single or multilayer core minitablet, a second active pharmaceutical ingredient containing layer coated thereon, and an enteric coating which dissolves at a pH of 3.5 or higher coated on the second active pharmaceutical ingredient containing layer. In this embodiment, the minitablet may further comprise an optional subcoating and/or functional coating between the first and second active pharmaceutical ingredient containing layers and/or the pH dependent enteric coating.

The active pharmaceutical ingredient may be the same in each active pharmaceutical ingredient containing layer of the present invention or it may be different.

In some embodiments, prior to applying the first and/or second enteric coatings, a subcoating and/or functional coating is applied either to avoid interactions of the active pharmaceutical ingredient with the enteric polymers or to control release of the active pharmaceutical ingredient, respectively.

In embodiments of the present invention comprising first and second enteric coatings, the enteric coatings may be the same or may be different. When coatings are the same, the difference in pH dissolution is controlled by the thickness of the enteric film coated on the minitablet.

By immediate release layer or layers as used herein it is meant that part of the core minitablet with a dissolution profile anywhere from 0 to 120 minutes in a suitable in vitro dissolution test. A suitable exemplary dissolution test may be, for example, dissolution carried out in 900 mL of phosphate buffer (pH 6.8) at temperature of 37.0° C.±0.5° C. using apparatus I (basket) rotating at a speed of 100 rpm. However, as will be understood by the skilled artisan upon reading this disclosure, variations on this test as well as the apparatus and conditions well known to those skilled in the art can be used.

Various methods for preparation of immediate release layers and the vehicles therein are well-known in the art. Generally recognized compendiums of such methods and ingredients include Remington: The Science and Practice of Pharmacy, Alfonso R. Gennaro, editor, 20th ed. Lippincott Williams & Wilkins: Philadelphia, Pa., 2000 and Sheth et al. Compressed Tablets, in Pharmaceutical Dosage Forms: Tablets, Vol 1. edited by H. A. Lieberman and L. Lachman, Dekker N.Y. (1980).

The immediate release layer or layers of a minitablet of the present invention can be prepared by direct compression of a mixture of the active pharmaceutical ingredient with a suitable carrier or excipient, such as carbohydrate or protein fillers, such as sugars, including lactose, sucrose, mannitol, or sorbitol; starch from corn, wheat, rice, potato, or other plants; cellulose, such as methyl cellulose, hydroxypropyl methyl-cellulose, sodium carboxymethylcellulose, or microcrystalline cellulose; gums including arabic and tragacanth; proteins such as gelatin and collagen; inorganics, such as kaolin, calcium carbonate, dicalcium phosphate, sodium chloride; magnesium carbonate; magnesium oxide; and other agents such as acacia and alginic acid.

Agents that facilitate disintegration and/or solubilization can also be added, such as the cross-linked polyvinyl pyrrolidone, sodium starch glycolate, Croscarmellose Sodium, alginic acid, or a salt thereof, such as sodium alginate, microcrystalline cellulose and corn starch.

Tablet binders that can be used include acacia, methylcellulose, sodium carboxymethylcellulose, polyvinylpyrrolidone (povidone), hydroxypropyl cellulose, hydroxypropyl methylcellulose, sucrose, starch and ethylcellulose.

Lubricants that can be used include magnesium stearates, stearic acid, sodium Stearyl fumerate, talc, waxes, oils, silicon dioxide and colloidal silica.

Fillers, agents that facilitate disintegration and/or solubilization, tablet binders and lubricants, including the aforementioned, can be used singly or in combination.

The immediate release layer or layers of the minitablets are then formulated, for example, by preparing a powder mixture by dry blending or granulating or slugging, adding a disintegrant and lubricant and pressing into minitablet layers.

By modified release layer or layers as used herein it is meant that part of the minitablet with a dissolution profile which is extended, delayed or controlled as compared to the immediate release layer.

Various methods for preparation of modified release layers and the vehicles therein are well-known in the art. Generally recognized compendiums of such methods and ingredients include Remington: The Science and Practice of Pharmacy, Alfonso R. Gennaro, editor, 20th ed. Lippincott Williams & Wilkins: Philadelphia, Pa., 2000 and Sheth et al. Compressed Tablets, in Pharmaceutical Dosage Forms: Tablets, Vol 1. edited by H. A. Lieberman and L. Lachman, Dekker N.Y. (1980).

A modified release layer or layers of a minitablet of the present invention can be prepared by incorporating release retarding excipients into the above-described formulation for the immediate release active pharmaceutical ingredient containing layer, and either completely omitting or reducing the amount of disintegrants.

Examples of release retarding excipients include, but are not limited to hydrophilic polymers such as hydroxypropylmethylcellulose, hydroxymethylcellulose, hydroxypropylcellulose and hydroxyethylcellulose, and which swell in contact with aqueous liquids, and control release of the drug by diffusion through the swollen polymer network.

Examples of other release retarding excipients include, but are not limited to, waxes such as carnauba wax, bees wax stearic acid and gums such as acacia, acrylic polymers, shellac, zein, polyvinylpyrrolidine including crosslinked polyvinylpyrrolidinone, vinyl acetate copolymers, polyethylene oxides, polyvinyl alcohols, and combinations comprising at least one of the foregoing materials.

Optionally, enteric polymers can also be added as release retarding agents to modify the release rates in certain pH environments. Examples of enteric polymers include, but are not limited to, polymers such as methacrylic acid-ethyl acrylate copolymer (1:1), ethacrylic acid-methyl methacrylate copolymer (1:1), methacrylic acid-methyl methacrylate copolymer (1:2), polyvinyl acetate phthalate (PVAP), hydroxypropyl methylcellulose acetate succinate (HPMCAS) and cellulose acetate phthalate (CAP).

The modified release layer or layers of the minitablets are formulated, for example, by preparing the powder mixture of drug or drugs with release retarding excipients by dry blending or granulating or slugging, adding a lubricant and pressing the mixture into tablet layers.

The inert layer or layers may comprise any biocompatible compound or mixture of compounds. The inert layer may be soluble or insoluble, permeable or impermeable, pH dependent or pH independent or any combination thereof depending upon the drug or drugs to be orally administered and/or the release mechanism required. Preferably, the inert layers are inert, insoluble and impermeable to drug in the drug containing layer. Accordingly, the inert layer preferably comprises no drug or drug in an amount which does not significantly modify bioequivalence.

Exemplary biocompatible materials for use in the inert layer include, but not limited to, waxes, polymers, gums and other pharmaceutically acceptable excipients either alone or in combination.

Exemplary wax excipients include, but are not limited to, wax and wax-like excipients such as carnauba wax, vegetable wax, fruit wax, microcrystalline wax, bees wax (white or bleached, and yellow), hydrocarbon wax, paraffin wax, cetyl esters wax or a combination comprising at least one of the foregoing waxes. Other suitable wax excipients include, for example, fatty alcohols (such as lauryl, myristyl, stearyl, cetyl or specifically cetostearyl alcohol), hydrogenated vegetable oil, hydrogenated castor oil, fatty acids such as stearic acid, fatty acid esters including fatty acid glycerides (mono-, di-, and tri-glycerides), polyethylene glycol (PEG) having a molecular weight of greater than about 3000 number average molecular weight, Mₙ (e.g. PEG 3350, PEG 4000, PEG 4600, PEG 6000, and PEG 8000), or a combination comprising at least one of the foregoing.

Exemplary polymer excipients include, for example acrylic polymers, alkylcelluloses including substituted alkylcelluloses, shellac, zein, polyvinylpyrrolidine including crosslinked polyvinylpyrrolidinone, vinyl acetate copolymers, polyethylene oxides, polyvinyl alcohols, and combinations comprising at least one of the foregoing materials.

Suitable acrylic polymers that can be used in the inert layer include, but are not limited to, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, or a combination comprising at least one of the foregoing polymers.

Suitable alkylcelluloses and substituted alkyl celluloses include, but are not limited to, methyl cellulose, ethylcellulose, hydroxy or carboxy substituted alkyl celluloses (e.g., hydroxyl propylcellulose, crosslinked hydroxypropylcellulose, carboxymethylcellulose, crosslinked sodium carboxymethylcellulose), hydroxy substituted alkyl-alkyl celluloses (e.g., hydroxypropylmethylcellulose), or a combination comprising at least one of the foregoing.

Exemplary additional pharmaceutically acceptable excipients for use in the inert layer include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, magnesium aluminum silicate (Veegum), and larch arabogalactan); alginates; polyethylene oxide; inorganic calcium salts; silicic acid; and combinations thereof.

Fillers, tablet binders and lubricants, including the aforementioned, can be used in the inert layer singly or in combination.

The inert layer of the minitablet of the present invention is formulated, for example, by preparing a powder mixture by dry blending or granulating or slugging, adding a lubricant and pressing into minitablet layers.

In one embodiment of the present invention, one or more of the immediate release layers and/or one or more of the modified release layers and optionally, one or more of the inert layers are compressed together to form a single core minitablet for a multilayer minitablet of the present invention.

In this embodiment, the core minitablet is then coated with an enteric polymer. Examples of enteric polymers include, but are not limited to, polymers such as methacrylic acid-ethyl acrylate copolymer (1:1), ethacrylic acid-methyl methacrylate copolymer (1:1), methacrylic acid-methyl methacrylate copolymer (1:2), polyvinyl acetate phthalate (PVAP), hydroxypropyl methylcellulose acetate succinate (HPMCAS) and cellulose acetate phthalate (CAP). Additionally, dyestuffs or pigments can be added to the enteric polymer coating for product identification or to characterize the quantity of active compound, i.e., dosage. Further, the enteric polymer can be optionally modified to include a pore-forming agent thereby resulting in a semi-enteric coating.

In some embodiments, prior to applying the enteric polymer coating, the core minitablet is coated with a subcoating or non-functional coating and then coated with the enteric polymer coating to avoid interactions of the active pharmaceutical ingredient with the enteric polymer. By “non-functional coating” it is meant a coating that does not significantly modify the release properties of the total formulation, for example, a cosmetic coating or an interlayer coating used to separate a functional coating from other components of the formulation. Examples of subcoating materials include, but are not limited to, film forming polymers like hydroxyalkyl celluloses such as hydroxymethyl cellulose, hydroxyethyl cellulose, hydroxypropyl cellulose and hydroxybutylcellulose, hydroxyalkyl alkylcelluloses such as hydroxyethyl methylcellulose and hydroxypropyl methylcellulose, polyvinylalcohols, polyvinylpyrrolidones, copolymers of polyvinylpyrrolidone with vinyl acetate, and combinations thereof.

In some embodiments, prior to applying the enteric polymer coating, the core minitablet is coated with a functional coatings or films. By “functional coating or film” it is meant a coating that modifies the release properties of the formulation. Examples of such coatings or films include, but are not limited to, controlled release, delayed release, modified release, pH independent coatings, and any combinations thereof.

The functional coating material can be in the form of a film coating comprising a solution or dispersion or a compressible powder mixture of a hydrophilic or hydrophobic polymer. Solvents used for application of the functional coating include pharmaceutically acceptable solvents, such as water, methanol, ethanol, methylene chloride, and a combination comprising at least one of the foregoing solvents.

Examples of functional coating materials include, but are not limited to, film forming polymers such as an alkylcellulose including methylcellulose or ethylcellulose, a hydroxyalkylcellulose such as hydroxymethylcellulose, hydroxyethylcellulose, hydroxypropylcellulose and hydroxybutylcellulose, a hydroxyalkyl alkylcellulose such as hydroxyethyl methylcellulose and hydroxypropyl methylcellulose, a carboxyalkylcellulose such as carboxymethylcellulose, an alkali metal salt of carboxyalkylcelluloses such as sodium carboxymethylcellulose, a carboxyalkyl alkylcellulose such as carboxymethyl ethylcellulose, a carboxyalkylcellulose ester, a starch, a pectin such as sodium carboxymethylamylopectine, a chitin derivate such as chitosan, a polysaccharide such as alginic acid, alkali metal and ammonium salts thereof, a carrageenan, a galactomannan, traganth, agar-agar, gum arabicum, guar gum and xanthan gum, a polyacrylic acid and the salts thereof, a polyvinylalcohol, a polyvinylpyrrolidone, a copolymer of polyvinylpyrrolidone with vinyl acetate, a polyalkylene oxide such as polyethylene oxide and polypropylene oxide and a copolymer of ethylene oxide and propylene oxide, or a combination comprising at least one of the foregoing.

The functional coating may optionally comprise a plasticizer, an additional film-former, a pore former, or a combination comprising at least one of the foregoing.

In another embodiment, a first active pharmaceutical ingredient is compressed into a single or multilayer tablet. In one embodiment, a second active pharmaceutical ingredient containing layer is then coated on the single or multilayer core minitablet. In an alternative embodiment, a first enteric coating which dissolves at a pH of 6.8 or higher is coated on the core minitablet followed by a second active pharmaceutical ingredient containing layer coated on the first enteric coating. In both embodiments, an enteric coating which dissolves at a pH of 3.5 or higher is then coated on the second active pharmaceutical ingredient containing layer.

In these embodiments, the minitablet may further comprise an optional subcoating and/or functional coating between the active pharmaceutical ingredient containing layers and the pH dependent enteric coatings.

While any active pharmaceutical ingredient administered orally can be formulated in accordance with the present invention, these multilayer minitablets provide a particularly useful formulation for the following drug categories:

Acid-labile pharmaceutical ingredients requiring protection against acidic stomach secretions such as proton pump inhibitors (PPIs). Examples of proton pump inhibitors include, but are not limited to omeprazole, lansoprazole, dexlansoprazole, esomeprazole, rabeprazole, pantoprazole, pariprazole, tenatoprazole and leminoprazole;

Drugs that cause upset or irritation to the upper GI tract such as, but not limited to, certain anti-depressants, selective serotonin reuptake inhibitors (SSRIs), non-steroidal anti-inflammatory drugs (NSAIDs) and aspirin; and

Drugs that are targeted for local delivery in the small intestine and/or large intestine and/or colon such as mesalamine and budesonide.

The active pharmaceutical ingredient may be the same in each active pharmaceutical ingredient containing layer of the present invention or it may be different.

A plurality of minitablets of the present invention is then encapsulated into a capsule for oral administration to a subject.

The following nonlimiting examples are provided to further illustrate the present invention.

### EXAMPLES

### Example 1

### Preparation of Enteric-Coated Multilayer Minitablets with 20% Omeprazole in Immediate Release Layer and 80% Omeprazole in Extended Release Layer

The immediate release layer contained Omeprazole magnesium (4.49 mg/capsule), microcrystalline cellulose (38.51 mg/capsule), lactose anhydrous (50.00 mg/capsule), hydroxypropyl cellulose (3.00 mg/capsule), croscarmellose sodium (3.00 mg/capsule), and magnesium stearate (1.00 mg/capsule).

The extended release layer contained Omeprazole magnesium (17.96 mg/capsule), microcrystalline cellulose (100.04 mg/capsule), lactose anhydrous (50.00 mg/capsule), hydroxypropyl cellulose (30.00 mg/capsule), and magnesium stearate (2.00 mg/capsule).

The subcoating contained Opadry II Clear (10 mg/capsule) and purified water which was removed during processing.

The enteric coating contained Eudragit L30D55 (24.32 mg/capsule), triethyl citrate (2.66 mg/capsule), talc (14.62 mg/capsule) and purified water which was removed during processing.

The immediate release layer and the extended release layer were prepared as follows:

Omeprazole magnesium was dry blended with all the ingredients except magnesium stearate for five minutes in a blender. Magnesium stearate was screened and then added to the blender. The mixture was then blended for another 2 minutes.

The layers were then compressed into bi-layer Minitablets of 15 mg each comprising 5 mg immediate release layer and 10 mg of extended release layer using a bi-layer tablet press.

The subcoating was prepared by dissolving Opadry II Clear in purified water and sprayed as a coating solution onto the bi layer minitablets in a wurster equipped fluid bed apparatus.

The enteric coating was prepared by mixing Eudragit L30D55 and triethyl citrate in a container using a mixer. In a separate container purified water was mixed with talc using mixer until the talc is evenly dispersed in the water. The talc suspension was then added to the Eudragit dispersion and mixed for 15 minutes. The resulting dispersion was mixed during the entire coating process. Using the wurster equipped fluid bed apparatus, the Eudragit/Talc dispersion was sprayed onto the sub-coated bi layer minitablets until the required weight gain was achieved.

### Example 2

### Preparation of Enteric-Coated Multilayer Minitablets with 70% Lansoprazole in Immediate Release Layer that Releases at pH Beyond 6.8 and 30% Lansoprazole in Immediate Release Layer that Releases at pH Beyond 5.5

The first immediate release layer that releases at pH beyond 6.8 contained Lansoprazole (21 mg/capsule), microcrystalline cellulose (108 mg/capsule), lactose anhydrous (60.00 mg/capsule), hydroxypropyl cellulose (6.00 mg/capsule), croscarmellose sodium (3.00 mg/capsule) and magnesium stearate (2.00 mg/capsule).

The second immediate release layer that releases at pH beyond 5.5 contained Lansoprazole (9 mg/capsule), hydroxypropyl methyl cellulose (3.00 mg/capsule) and Sodium Lauryl sulfate (0.10 mg/capsule. Purified water was removed during processing.

The subcoating contained Opadry II Clear (10 mg/capsule) and purified water which was removed during processing.

The first enteric coating that dissolves at pH beyond 6.8 contained Eudragit L100 (24.32 mg/capsule), triethyl citrate (2.66 mg/capsule), talc (14.62 mg/capsule) and SD3A alcohol which was removed during processing.

The second enteric coating that dissolves at pH beyond 5.5 contained Eudragit L30D55 (24.32 mg/capsule), triethyl citrate (2.66 mg/capsule), talc (14.62 mg/capsule) and purified water which was removed during processing.

The first immediate release layer that releases at pH beyond 6.8 was prepared as follows:

Lansoprazole was dry blended with all the ingredients except magnesium stearate for five minutes in a blender. Magnesium stearate was screened and then added to the blender. The mixture was then blended for another 2 minutes.

The blend was then compressed into Minitablets of 10 mg each using tablet press fitted with multi tip tooling.

The subcoating was prepared by dissolving Opadry II Clear in purified water and sprayed as a coating solution onto the minitablets in a wurster equipped fluid bed apparatus.

The first enteric coating was prepared by dissolving Eudragit L100 in SD3 Alcohol and mixing triethyl citrate in a container using a mixer. The talc was then added to the Eudragit solution and mixed for 15 minutes. The resulting dispersion was mixed during the entire coating process. Using the wurster equipped fluid bed apparatus, the Eudragit/Talc dispersion was sprayed onto the sub-coated minitablets until the required weight gain was achieved.

Then once again subcoating was applied to the enteric coated Minitablets.

The second immediate release drug layering was carried out using the wurster equipped fluid bed apparatus. Lansoprazole was sprayed onto the enteric coated Minitablets from a water suspension containing the dissolved binder and the wetting agent.

Then once again subcoating was applied to the drug layered Minitablets.

The second enteric coating was prepared by mixing Eudragit L30D55 and triethyl citrate in a container using a mixer. In a separate container purified water was mixed with talc using mixer until the talc is evenly dispersed in the water. The talc suspension was then added to the Eudragit dispersion and mixed for 15 minutes. The resulting dispersion was mixed during the entire coating process. Using the wurster equipped fluid bed apparatus, the Eudragit/Talc dispersion was sprayed onto the sub-coated minitablets until the required weight gain was achieved.

### Example 3

### Preparation of Enteric-Coated Multilayer Minitablets with 20% Paroxetine HCl in Immediate Release Layer and 80% Paroxetine HCl in Extended Release Layer

The immediate release layer contained Paroxetine HCl (7.5 mg/capsule), microcrystalline cellulose (35.50 mg/capsule), lactose anhydrous (50.00 mg/capsule), hydroxypropyl cellulose (3.00 mg/capsule), croscarmellose sodium (3.00 mg/capsule), and magnesium stearate (1.00 mg/capsule).

The extended release layer contained Paroxetine HCl (30 mg/capsule), microcrystalline cellulose (68.00 mg/capsule), lactose anhydrous (50.00 mg/capsule), hydroxypropyl cellulose (50.00 mg/capsule), and magnesium stearate (2.00 mg/capsule).

The subcoating contained Opadry II Clear (10 mg/capsule) and purified water which was removed during processing.

The enteric coating contained Cellulose acetate phthalate (Aquacoat CPD 30D) (18.30 mg/capsule), hydroxypropyl methyl cellulose (7.80 mg/capsule), triethyl citrate (4.80 mg/capsule) and purified water which was removed during processing.

The immediate release layer and the extended release layer were prepared as follows:

Paroxetine HCl was dry blended with all the ingredients except magnesium stearate for five minutes in a blender. Magnesium stearate was screened and then added to the blender. The mixture was then blended for another 2 minutes.

The layers were then compressed into bi-layer Minitablets of 15 mg each comprising 5 mg immediate release layer and 10 mg of extended release layer using a bi-layer tablet press.

The subcoating was prepared by dissolving Opadry II Clear in purified water and sprayed as a coating solution onto the bi layer minitablets in a wurster equipped fluid bed apparatus.

The enteric coating was prepared by mixing Cellulose acetate phthalate (Aquacoat CPD 30D) and triethyl citrate in a container using a mixer. In a separate container purified water was mixed with hydroxypropyl methyl cellulose using mixer until a clear solution was obtained. The hydroxypropyl methyl cellulose solution was then added to the Cellulose acetate phthalate dispersion and mixed for 15 minutes. The resulting dispersion was mixed during the entire coating process. Using the wurster equipped fluid bed apparatus, the Cellulose acetate phthalate/hydroxypropyl methyl cellulose dispersion was sprayed onto the sub-coated bi layer minitablets until the required weight gain was achieved.

### Example 4

### Preparation of Enteric-Coated Multilayer Minitablets with 80% Paroxetine HCl in Extended Release Layer that Releases at pH Beyond 6.8 and 20% Paroxetine HCl in Immediate Release Layer that Releases at pH Beyond 3.5

The extended release layer contained Paroxetine HCl (30 mg/capsule), microcrystalline cellulose (68.00 mg/capsule), lactose anhydrous (50.00 mg/capsule), hydroxypropyl cellulose (50.00 mg/capsule), and magnesium stearate (2.00 mg/capsule).

The immediate release layer that releases at pH beyond 3.5 contained Paroxetine HCl (7.5 mg/capsule), hydroxypropyl methyl cellulose (3.00 mg/capsule) and Sodium Lauryl sulfate (0.10 mg/capsule. Purified water was removed during processing.

The subcoating contained Opadry II Clear (10 mg/capsule) and purified water which was removed during processing.

The first enteric coating that releases at pH beyond 6.8 contained Eudragit L100 (24.32 mg/capsule), triethyl citrate (2.66 mg/capsule), talc (14.62 mg/capsule) and SD3A alcohol which was removed during processing.

The second enteric coating that releases at pH beyond 3.5 contained Cellulose acetate phthalate (Aquacoat CPD 30D) (18.30 mg/capsule), hydroxypropyl methyl cellulose (7.80 mg/capsule), triethyl citrate (4.80 mg/capsule) and purified water which was removed during processing.

The extended release layer that releases at pH beyond 6.8 was prepared as follows:

Paroxetine HCl was dry blended with all the ingredients except magnesium stearate for five minutes in a blender. Magnesium stearate was screened and then added to the blender. The mixture was then blended for another 2 minutes.

The blend was then compressed into Minitablets of 10 mg each using tablet press fitted with multi tip tooling.

The subcoating was prepared by dissolving Opadry II Clear in purified water and sprayed as a coating solution onto the minitablets in a wurster equipped fluid bed apparatus.

The first enteric coating was prepared by dissolving Eudragit L100 in SD3 Alcohol and mixing triethyl citrate in a container using a mixer. The talc was then added to the Eudragit solution and mixed for 15 minutes. The resulting dispersion was mixed during the entire coating process.

Using the wurster equipped fluid bed apparatus, the Eudragit/Talc dispersion was sprayed onto the sub-coated minitablets until the required weight gain was achieved.

Then once again subcoating was applied to the enteric coated Minitablets.

The immediate release drug layering was carried out using the wurster equipped fluid bed apparatus. Paroxetine HCl was sprayed onto the enteric coated Minitablets from a water suspension containing the dissolved binder and the wetting agent.

Then once again subcoating was applied to the drug layered Minitablets.

The second enteric coating was prepared by mixing Cellulose acetate phthalate (Aquacoat CPD 30D) and triethyl citrate in a container using a mixer. In a separate container purified water was mixed with hydroxypropyl methyl cellulose using mixer until a clear solution was obtained. The hydroxypropyl methyl cellulose solution was then added to the Cellulose acetate phthalate dispersion and mixed for 15 minutes. The resulting dispersion was mixed during the entire coating process. Using the wurster equipped fluid bed apparatus, the Cellulose acetate phthalate/hydroxypropyl methyl cellulose dispersion was sprayed onto the sub-coated bi layer minitablets until the required weight gain was achieved.

1. A multilayer minitablet 5 mm or less in diameter comprising:
an immediate release layer containing an active pharmaceutical ingredient;
a modified release layer containing an active pharmaceutical ingredient compressed with or to said first immediate release layer to form a core minitablet; and
a pH dependent enteric coating around the compressed core minitablet.

2. The multilayer minitablet of claim 1 further comprising one or more optional inert layers.

3. The multilayer minitablet of claim 1 further comprising a subcoating and/or a functional coating between the multilayer core minitablet and the pH dependent enteric coating.

4. The multilayer minitablet of claim 1 wherein the active pharmaceutical ingredient containing layers comprise a single active pharmaceutical ingredient.

5. The multilayer minitablet of claim 1 wherein the active pharmaceutical ingredient containing layers comprise two or more active pharmaceutical ingredients.

6. The multilayer minitablet of claim 1 wherein the active pharmaceutical ingredient is selected from the group consisting of proton pump inhibitors, selective serotonin reuptake inhibitors and nonsteroidal antiinflammatory drugs.

7-8. (canceled)

9. The multilayer minitablet of claim 1 wherein the active pharmaceutical ingredient causes irritation to the upper gastrointestinal tract or is targeted for local delivery in the small intestine and/or large intestine and/or colon.

10. (canceled)

11. A capsule comprising a plurality of multilayer minitablets of claim 1.

12. A multilayer minitablet 5 mm or less in diameter comprising:
a first active pharmaceutical ingredient compressed into a single or multilayer core minitablet;
a first enteric coating which dissolves at a pH of 6.8 or higher on said core minitablet;
a second active pharmaceutical ingredient containing layer coated on said first enteric coating; and
a second enteric coating which dissolves at a pH of 3.5 or higher on said second active pharmaceutical ingredient containing layer.

13. The multilayer minitablet of claim 12 further comprising a subcoating and/or a functional coating between the active pharmaceutical ingredient containing layer and the pH dependent enteric coating.

14. The multilayer minitablet of claim 12 wherein the active pharmaceutical ingredient containing layers comprise a single active pharmaceutical ingredient.

15. The multilayer minitablet of claim 12 wherein the active pharmaceutical ingredient containing layers comprise two or more active pharmaceutical ingredients.

16. The multilayer minitablet of claim 12 wherein the active pharmaceutical ingredient is selected from the group consisting of proton pump inhibitors, selective serotonin reuptake inhibitors and nonsteroidal antiinflammatory drugs.

17-18. (canceled)

19. The multilayer minitablet of claim 12 wherein the active pharmaceutical ingredient causes irritation to the upper gastrointestinal tract or is targeted for local delivery in the small intestine and/or large intestine and/or colon.

20. (canceled)

21. The multilayer minitablet of claim 12 wherein the first and second enteric coatings are the same.

22. The multilayer minitablet of claim 12 wherein the first and second enteric coatings are different.

23. A capsule comprising a plurality of multilayer minitablets of claim 12.

24. A multilayer minitablet 5 mm or less in diameter comprising:
one or more immediate release layers compressed to form a core minitablet, each immediate release layer containing an active pharmaceutical ingredient; and
a pH dependent enteric coating around the compressed core minitablet.

25. The multilayer minitablet of claim 24 further comprising one or more optional inert layers.

26. The multilayer minitablet of claim 24 further comprising a subcoating and/or a functional coating between the multilayer core minitablet and the pH dependent enteric coating.

27. The multilayer minitablet of claim 24 comprising two or more immediate release layers wherein each immediate release layer comprises a different active pharmaceutical ingredient.

28. The multilayer minitablet of claim 24 wherein the active pharmaceutical ingredient is selected from the group consisting of proton pump inhibitors, selective serotonin reuptake inhibitors and nonsteroidal antiinflammatory drugs.

29-30. (canceled)

31. The multilayer minitablet of claim 24 wherein the active pharmaceutical ingredient causes irritation to the upper gastrointestinal tract or is targeted for local delivery in the small intestine and/or large intestine and/or colon.

32. (canceled)

33. A capsule comprising a plurality of multilayer minitablets of claim 24.

34. A multilayer minitablet 5 mm or less in diameter comprising:
a first active pharmaceutical ingredient compressed into a single or multilayer core minitablet;
a second active pharmaceutical ingredient containing layer coated on said single or multilayer core minitablet; and
an enteric coating which dissolves at a pH of 3.5 or higher on said second active pharmaceutical ingredient containing layer.

35. The multilayer minitablet of claim 34 further comprising a subcoating and/or a functional coating between the first active pharmaceutical ingredient containing layer and the second active pharmaceutical ingredient containing layer.

36. The multilayer minitablet of claim 34 further comprising a subcoating and/or a functional coating between the second active pharmaceutical ingredient containing layer and the pH dependent enteric coating.

37. The multilayer minitablet of claim 34 wherein the active pharmaceutical ingredient containing layers comprise a single active pharmaceutical ingredient.

38. The multilayer minitablet of claim 34 wherein the active pharmaceutical ingredient containing layers comprise two or more active pharmaceutical ingredients.

39. The multilayer minitablet of claim 34 wherein the active pharmaceutical ingredient is selected from the group consisting of proton pump inhibitors, selective serotonin reuptake inhibitors and nonsteroidal antiinflammatory drugs.

40-41. (canceled)

42. The multilayer minitablet of claim 34 wherein the active pharmaceutical ingredient causes irritation to the upper gastrointestinal tract or is targeted for local delivery in the small intestine and/or large intestine and/or colon.

43. (canceled)

44. A multilayer minitablet 5 mm or less in diameter comprising:
one or more modified release layers compressed to form a core minitablet, each modified release layer containing an active pharmaceutical ingredient; and
a pH dependent enteric coating around the compressed core minitablet.

45. The multilayer minitablet of claim 44 further comprising one or more optional inert layers.

46. The multilayer minitablet of claim 44 further comprising a subcoating and/or a functional coating between the multilayer core minitablet and the pH dependent enteric coating.

47. The multilayer minitablet of claim 44 comprising two or more modified release layers wherein each modified release layer comprises a different active pharmaceutical ingredient.

48. The multilayer minitablet of claim 44 wherein the active pharmaceutical ingredient is selected from the group consisting of proton pump inhibitors, selective serotonin reuptake inhibitors and nonsteroidal antiinflammatory drugs.

49-50. (canceled)

51. The multilayer minitablet of claim 44 wherein the active pharmaceutical ingredient causes irritation to the upper gastrointestinal tract or is targeted for local delivery in the small intestine and/or large intestine and/or colon.

52. (canceled)

53. A capsule comprising a plurality of multilayer minitablets of claim 44.
